(19) United States
(12) Patent Application Publication
(10) Pub. No.: US 2024/0390146 A1
(43) Pub. Date: Nov. 28, 2024

(54) A HEART VALVE HOLDER AND METHOD OF USE THEREOF

(71) Applicants: NATIONAL UNIVERSITY OF SINGAPORE, Singapore (SG); NATIONAL UNIVERSITY HOSPITAL ( SINGAPORE) PTE LTD, Singapore (SG)

(72) Inventor: Theodoros KOFIDIS, Singapore (SG)

(21) Appl. No.: 18/693,541

(22) PCT Filed: Sep. 22, 2022
(86) PCT No.: PCT/SG2022/050686

§ 371 Date: Mar. 20, 2024

(30) Foreign Application Priority Data
Sep. 24, 2021 (SG) 10202110635Q

(51) Int. Cl. A61F 2/24

(52) U.S. Cl. CPC A61F 2/2466; A61F 2/2448; A61F 2220/0008

(57) ABSTRACT

A heart valve holder for holding a prosthetic heart valve and a method for implanting the prosthetic heart valve are disclosed, wherein the heart valve holder comprises a hollow structure having a cross-section that mimics a shape of an annulus of the prosthetic heart valve, the hollow structure comprising an upper surface; a platform extending from at least one wall of the hollow structure, the platform comprising a first connector element; and at least one anchoring element formed at the upper surface of the hollow structure, the at least one anchoring element comprising at least one anchoring hole for suturing of the prosthetic heart valve to the heart valve holder.

### CROSS REFERENCE TO RELATED APPLICATIONS

The present application claims the benefit of the Singapore application Ser. No. 10202110635Q filed on 24 Sep. 2021, the disclosure of which is hereby incorporated in its entirety by reference herein.

### TECHNICAL FIELD

The present disclosure relates to a medical device to hold a prosthetic heart valve. In particular, the present disclosure relates to a heart valve holder to hold a prosthetic heart valve and method of use of the heart valve holder.

### BACKGROUND

In heart valve replacement surgery, an artificial valve, also known as a prosthetic heart valve, is attached to an annulus of the human heart to replace a native valve of the human heart that is not functioning correctly. A heart valve holder is used during the introduction or implantation of a new prosthetic heart valve during surgery for the patient's safety as it prevents contamination. The heart valve holder is used for mounting and carrying a prosthetic heart valve from its packaging into the heart for implantation. The heart valve holder also provides support to hold the prosthetic heart valve in the right place when surgeons are replacing the native heart valve.

Existing prosthetic heart valve holders are designed to be used in conjunction with existing biological and mechanical heart valve prostheses. Existing biological and mechanical heart valve prostheses have a rigid and circular annulus, and do not include cords. On the other hand, a natural human heart valve has an asymmetrical shape, is not rigid, and has chordae tendineae connecting the leaflets of the valve to papillary muscles.

U.S. Pat. No. 10,709,560 granted Jul. 14, 2020, and U.S. Patent Publication 2019/0321168 published Oct. 24, 2019, by the current applicants, the disclosure of which is incorporated herein by reference, describe a naturally designed prosthetic mitral heart valve. The naturally designed prosthetic mitral heart valve is designed to mimic a natural mitral heart valve and includes two flexible leaflets and an asymmetric and flexible ring, which can move with the natural distortion of the heart muscle during a cardiac cycle. Cords, similar to the native chordae tendineae of the patient, are included in the prosthetic mitral heart valve to mimic the natural prevention of backflow of blood into the atrium and to provide support to the left ventricle during systole. However, existing prosthetic heart valve holders are not suitable to be used with such a naturally designed prosthetic mitral heart valve designed to mimic a natural mitral heart valve due to their unnatural shape and inability to accommodate cords. In addition, the naturally designed prosthetic mitral heart valve needs to have minimal physical connection with any external products and should not have any contact where the prosthesis is to be implanted.

It is therefore desirable to provide an improved heart valve holder and valve deployment system for use with a prosthetic heart valve designed to mimic a natural heart valve.

### SUMMARY

The present disclosure discloses a heart valve holder, and a container to store the heart valve holder. The heart valve holder and container have several advantages over existing heart valve holders and containers. Firstly, the disclosed heart valve holder provides stable support, and may be placed in a container without any movement to prevent any damage when transported. The disclosed heart valve holder and container are easy to assemble for the manufacturer, and easy to disassemble for the surgeon during surgery. In addition, the disclosed heart valve holder does not come into physical contact with the part of the heart where the prosthetic mitral heart valve is to be attached.

There is provided according to an embodiment of the disclosure, a heart valve holder for holding a prosthetic heart valve, the heart valve holder comprising: a hollow structure having a cross-section that mimics a shape of an annulus of the prosthetic heart valve, the hollow structure comprising an upper surface; a platform extending from at least one wall of the hollow structure, the platform comprising a first connector element; and at least one anchoring element formed at the upper surface of the hollow structure, the at least one anchoring element comprising at least one anchoring hole for suturing the prosthetic heart valve to the heart valve holder. Optionally, the cross-section may be D-shaped to mimic the shape of the annulus of the prosthetic heart valve, the annulus of the prosthetic heart valve mimicking a shape of a mitral valve annulus. Optionally, the heart valve holder may be dimensioned to fit within the prosthetic heart valve. Optionally, the hollow structure may be a tapered tube, the tapered tube tapering from a top section of the hollow structure to a bottom section of the hollow structure. Optionally, the at least one anchoring element may be positioned corresponding to an anterolateral commissure, a posteromedial commissure, a midpoint of a posterior leaflet, or a midpoint of an anterior leaflet.

According to some embodiments, the heart valve holder may further comprise at least one leg at the bottom section of the hollow structure, the at least one leg comprising at least one anchoring hole for suturing of the prosthetic heart valve to the heart valve holder. Optionally, the at least one leg may be positioned corresponding to a papillary muscle of a left ventricle of a heart.

According to some embodiments, the heart valve holder may further comprise a handle, the handle comprising a second connector element to be connected to the first connector element. Optionally, the handle may comprise a rod. Optionally, the handle may comprise a flexible section. Optionally, the handle may comprise a ruler section. Optionally, the second connector element may comprise a threaded portion for attachment to the first connector element. Optionally, the platform may further comprise a third connector element. Optionally, the heart valve holder may further comprise a support structure, the support structure comprising a fourth connector element to be connected to the third connector element. Optionally, the fourth connector element may comprise a threaded top for attachment to the third connector element. Optionally, the fourth connector element may comprise an indent to receive the third connector element. Optionally, the third connector element may comprise extended arms for securing the fourth connector element.

There is further provided according to an embodiment of the disclosure, a method of implanting a prosthetic heart valve, the method comprising: providing a heart valve holder for holding a prosthetic heart valve, the heart valve holder comprising: a hollow structure having a cross-section that mimics a shape of an annulus of the prosthetic heart valve, the hollow structure comprising an upper surface; a platform extending from at least one wall of the hollow structure, the platform comprising a first connector element; and at least one anchoring element formed at the upper surface of the hollow structure, the at least one anchoring element comprising at least one anchoring hole for suturing the prosthetic heart valve to the heart valve holder; providing a handle, the handle comprising a second connector element; connecting the handle to the heart valve holder; mounting the prosthetic heart valve on the heart valve holder; attaching at least one suture wire between a heart and the prosthetic heart valve; and sliding the prosthetic heart valve along the at least one suture wire into the heart. Optionally, mounting the prosthetic heart valve may comprise using a single suture to attach the prosthetic heart valve to the heart valve holder. Optionally, the method may further comprise using a ruler portion on the handle to take measurements of the heart. Optionally, the method may further comprise positioning the prosthetic heart valve in the heart using marked landmarks on a native valve annulus; and tying the at least one suture wire to attach the prosthetic heart valve to the heart.

### BRIEF DESCRIPTION OF THE DRAWINGS

In order for the present disclosure, to be better understood and for its practical applications to be appreciated, the following Figures are provided and referenced hereafter. It should be noted that the Figures are given as examples only and in no way limit the scope of the invention.

FIG. 1 schematically illustrates an apparatus for holding and transporting a prosthetic heart valve, in accordance with some embodiments of the present disclosure;

FIG. 2A is a schematic illustration of a front perspective view of a heart valve holder, and FIG. 2B is a bottom perspective view of a heart valve holder, in accordance with some embodiments of the present disclosure;

FIG. 3A is a schematic illustration of a top view of a prosthetic heart valve mounted on a heart valve holder, and FIG. 3B is a schematic illustration of a front perspective view of a prosthetic heart valve mounted on a heart valve holder, in accordance with some embodiments of the present disclosure;

FIG. 4 is a schematic illustration of a tapering of a heart valve holder, in accordance with some embodiments of the present disclosure;

FIG. 5 is a schematic illustration of a vertical cross-section of a first alternative heart valve holder, in accordance with some embodiments of the present disclosure;

FIG. 6 is a schematic illustration of a top perspective view of a second alternative heart valve holder, in accordance with some embodiments of the present disclosure;

FIG. 7A is a schematic illustration of a front perspective view of a third alternative heart valve holder, in accordance with some embodiments of the present disclosure;

FIG. 7B is a schematic illustration of a top view of a third alternative heart valve holder;

FIG. 7C is a schematic illustration of a vertical cross-section (section D-D) of a third alternative heart valve holder, in accordance with some embodiments of the present disclosure;

FIG. 8 is a schematic illustration of a handle, in accordance with some embodiments of the present disclosure;

FIG. 9 is a schematic illustration of a first alternative handle, in accordance with some embodiments of the present disclosure;

FIG. 10 is a schematic illustration of a second alternative handle, in accordance with some embodiments of the present disclosure;

FIG. 11 is a schematic illustration of a third alternative handle, in accordance with some embodiments of the present disclosure;

FIG. 12 is a schematic illustration of a support structure to support heart valve holder, in accordance with some embodiments of the present disclosure;

FIG. 13 is a schematic illustration of a support structure supporting a heart valve holder, in accordance with some embodiments of the present disclosure;

FIG. 14 is a schematic illustration of a support structure and a heart valve holder inserted into a container for transport, in accordance with some embodiments of the present disclosure;

FIG. 15A is a schematic illustration of a first alternative support structure supporting a heart valve holder, and FIG. 15B is a schematic illustration of a first alternative support structure, in accordance with some embodiments of the present disclosure;

FIG. 16A is a schematic illustration of a front perspective view of a second alternative support structure for supporting a heart valve holder, in accordance with some embodiments of the present disclosure;

FIG. 16B is a schematic illustration of a vertical cross-section of a second alternative support structure with a heart valve holder shown being retained by the second alternative support structure;

FIG. 16C is a schematic illustration of a vertical cross-section of a second alternative support structure with a heart valve holder shown with the tip of the handle;

FIG. 16D is a schematic illustration of a vertical cross-section of a second alternative support structure with a heart valve holder shown having extended arms to be disengaged from the second alternative support structure, in accordance with some embodiments of the present disclosure;

FIG. 16E is a schematic illustration of a vertical cross-section of a second alternative support structure with a heart valve holder shown being detached from the second alternative support structure, in accordance with some embodiments of the present disclosure; and

FIGS. 17A, 17B, 17C, 17D and 17E are schematic illustrations of a process of implanting a prosthetic heart valve in a heart of a patient using a heart valve holder, in accordance with some embodiments of the present disclosure.

Identical or duplicate or equivalent or similar structures, elements, or parts that appear in one or more drawings are generally labelled with the same reference numeral, optionally with an additional letter or letters to distinguish between similar entities or variants of entities and may not be repeatedly labelled and/or described. References to previously presented elements are implied without necessarily further citing the drawing or description in which they appear.

### DETAILED DESCRIPTION

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, use of the conjunction “or” as used herein is to be understood as inclusive (any or all of the stated options).

With specific reference now to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

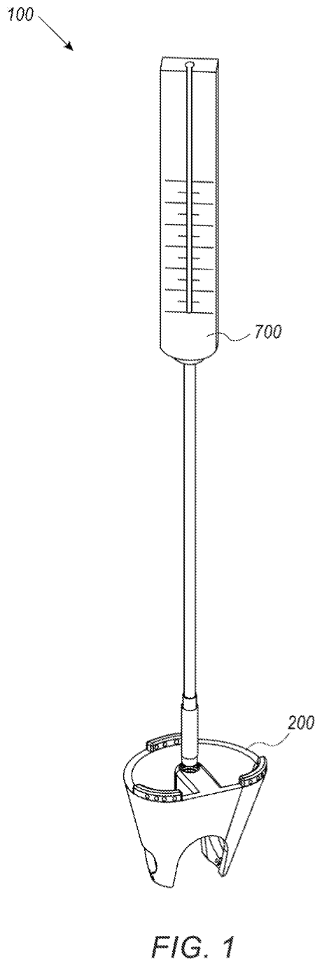

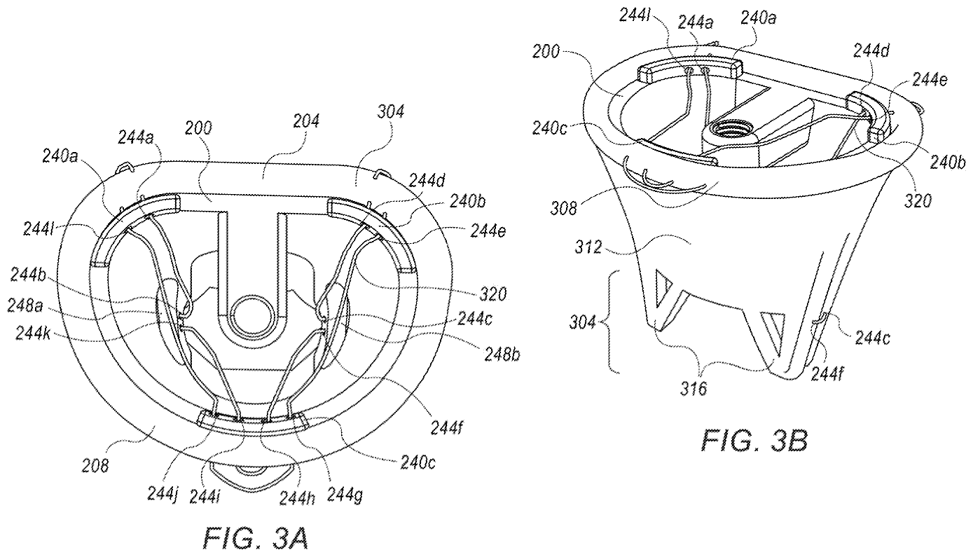

FIG. 1 schematically illustrates an apparatus 100 for holding and transporting a prosthetic heart valve, in accordance with some embodiments of the present disclosure. Apparatus 100 is used by an operator to mount and carry a prosthetic heart valve. Apparatus 100 may be used to mount and carry a prosthetic heart valve from its packaging to a heart for implantation. Apparatus 100 may comprise a heart valve holder 200 and handle 700. Heart valve holder 200 and handle 700 may be removably attached to each other using a screw mechanism or any other appropriate mechanism. In some embodiments, heart valve holder disclosed in the present disclosure including heart valve holder 200 may be designed such that it may be suitable for holding any prosthetic heart valve, including a bioprosthetic heart valve and a mechanical heart prosthetic heart valve. In some embodiments, the prosthetic heart valve may be a mitral valve prosthesis that mimics a native mitral valve, the mitral valve prosthesis having an annulus that mimics the shape of the native mitral valve annulus. In some embodiments, the prosthetic heart valve may be mounted onto heart valve holder 200 using suture threads such as a single safety suture 320 (see FIGS. 3A and 3B). In some embodiments, an operator may attach handle 700 to heart valve holder 200 and hold the handle 700 to transport heart valve holder 200 with the prosthetic heart valve mounted. Handle 700 allows a surgeon or user to control heart valve holder 200 to deliver, handle and implant the prosthetic heart valve with precision.

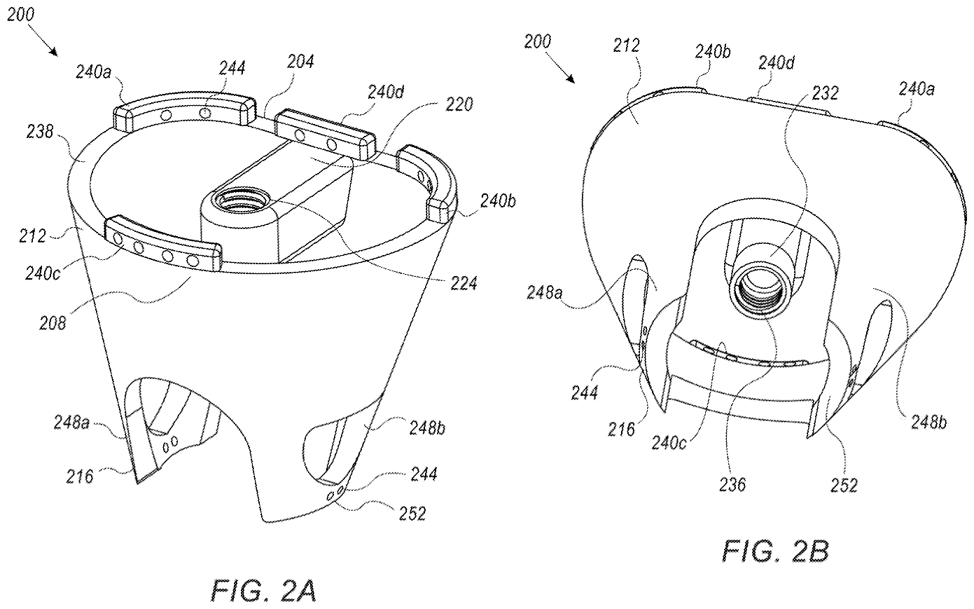

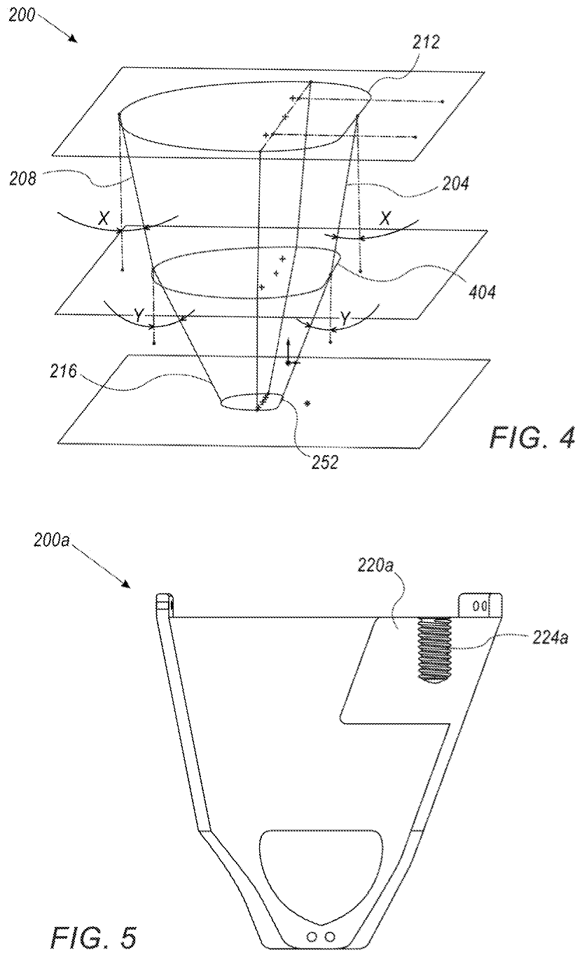

FIG. 2A is a schematic illustration of a front perspective view of a heart valve holder 200, and FIG. 2B is a bottom perspective view of a heart valve holder 200, in accordance with some embodiments of the present disclosure. Heart valve holder 200 holds a prosthetic heart valve for transportation to the heart for implantation during heart valve replacement surgery. Heart valve holder 200 may be made of materials such as polypropylene, polyetherketoneketone, acrylonitrile butadiene styrene or ABS and Delrin (a synthetic polymer or polyoxymethylene from DuPont). In some embodiments, said polypropylene, polyetherketoneketone, ABS and Delrin (polyoxymethylene) may be of a medical grade. In some embodiments, said polypropylene, polyetherketoneketone, ABS and Delrin (polyoxymethylene) may be biocompatible polypropylene, polyetherketoneketone, ABS and Delrin (polyoxymethylene). In some embodiments, said biocompatible materials may comply with the US Pharmacopeial Convention (USP) Class VI or ISO 10993-1. Heart valve holder 200 is a hollow structure with a cross-section which mimics a shape of an annulus 308 of a prosthetic heart valve 304 (see FIGS. 3A and 3B). In some embodiments, heart valve holder 200 may have a cross-section which mimics a shape of a prosthetic heart valve annulus, such prosthetic heart valve annulus mimicking the shape of a native heart valve annulus. In some embodiments, where heart valve holder 200 is configured to hold a mitral valve prosthesis, heart valve holder 200 may have a D-shaped cross-section to mimic the shape of a mitral valve prosthesis annulus that mimics the shape of the native mitral valve annulus. In some embodiments, heart valve holder 200 may have a cross-section that is odd-shaped. In some embodiments, heart valve holder 200 may be a hollow tapered tube with a horizontal cross-section that mimics the general shape of an annulus of a prosthetic heart valve, the prosthetic heart valve having an annulus that mimics the shape of the native heart valve annulus. In some embodiments, the heart valve holder 200 may comprise an anterior side 204 mimicking an anterior annulus of a mitral valve prosthesis having an annulus that mimics the shape of the native mitral valve annulus and a posterior side 208 mimicking a posterior annulus of a mitral valve prosthesis having an annulus that mimics the shape of the native mitral valve annulus, tapering from a top section 212 of heart valve holder 200 to a bottom section 216 of heart valve holder 200. A midpoint of anterior side 204 may be defined as a 12 o'clock position of the heart valve holder 200 and a midpoint of posterior side 208 may be defined as a 6 o'clock position of the heart valve holder 200 when orientating heart valve holder 200. In some embodiments, the cross-section of heart valve holder 200 may taper evenly between top section 212 and bottom section 216. In other embodiments, the cross-section of heart valve holder 200 may taper at varying gradients between top section 212 and bottom section 216 (see FIG. 4). In some embodiments, heart valve holder 200 may be dimensioned to fit within a prosthetic heart valve 304 (see FIGS. 3A and 3B), such that the prosthetic heart valve 304 may be suspended outside the heart valve holder 200 when secured to the heart valve holder 200. In some embodiments, heart valve holder 200 may have a height of between 20 and 30 mm, and preferably 28 mm, from top section 212 to bottom section 216. In some embodiments, heart valve holder 200 may have a wall thickness of between 0.5 and 2 mm, and preferably 1 mm. In some embodiments, the cross-section of top section 212 of heart valve holder 200 for a mitral valve prosthesis that mimics a native mitral valve may have a width that mimics the anterolateral posteromedial diameter distance of a mitral valve and a length that mimics the anterior-posterior diameter of a mitral valve annulus. In some embodiments, the top section 212 may have a width of between 18 and 30 mm, and preferably 25 mm. In some embodiments, heart valve holder 200 may have a width of 28 mm at top section 212 for a heart valve sized CC36 and a width of 25 mm for a heart valve sized CC32. In some embodiments, heart valve holder 200 may have a length of between 20 and 35 mm, and preferably 26 mm.

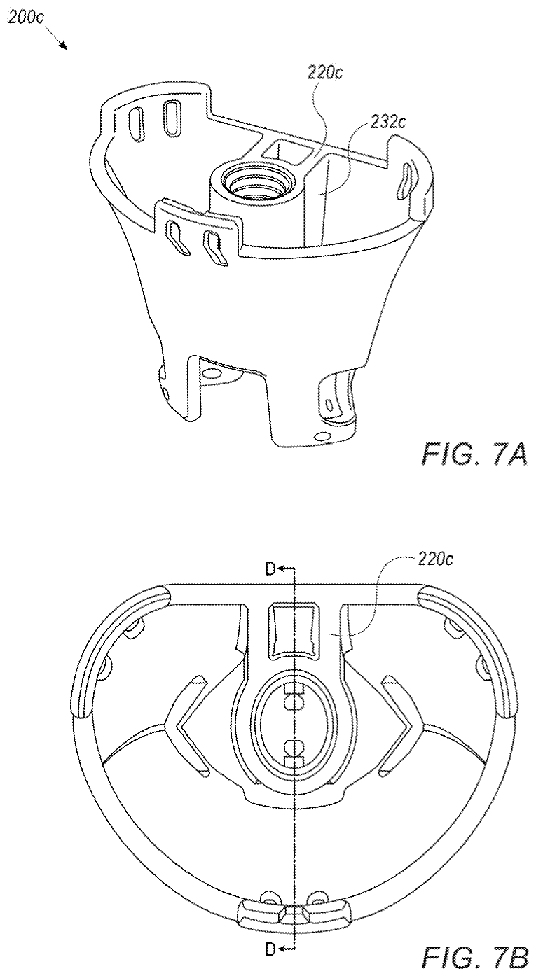

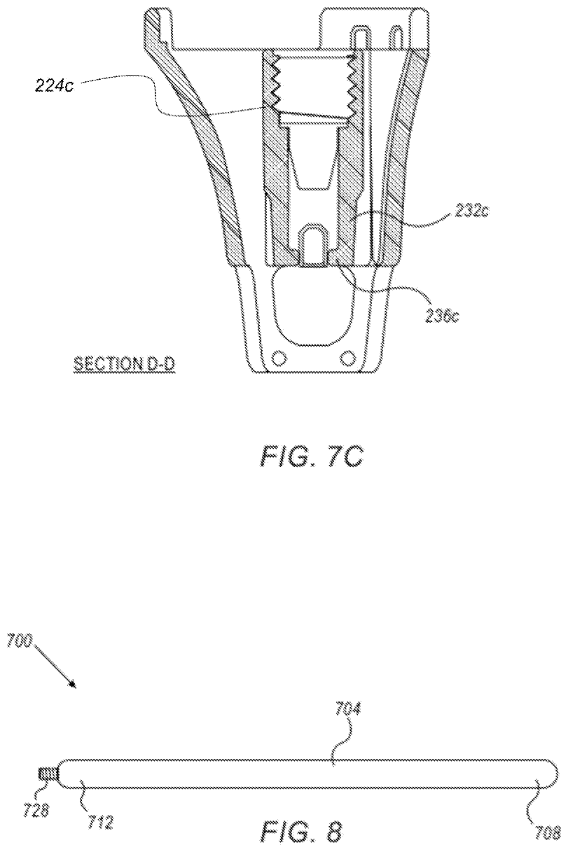

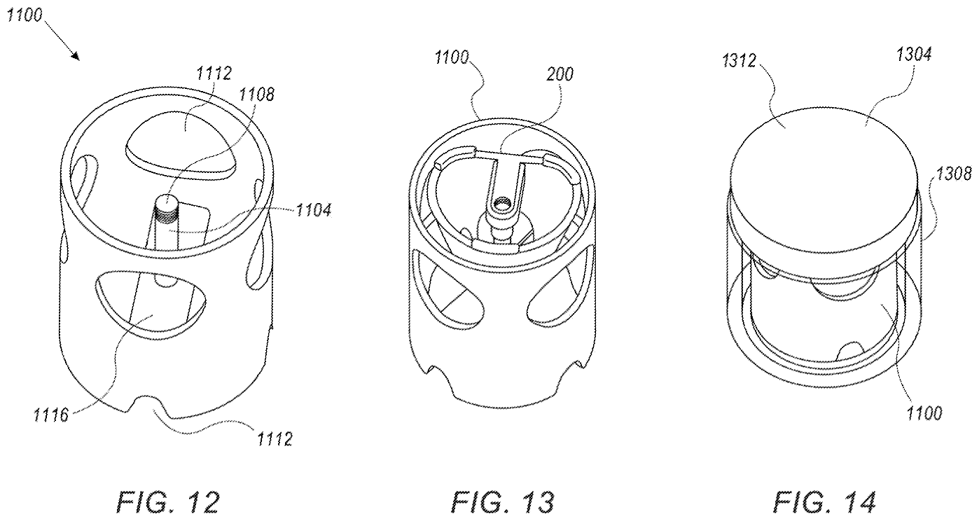

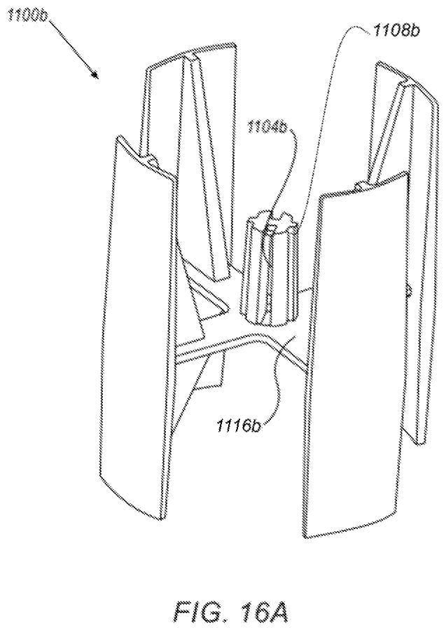

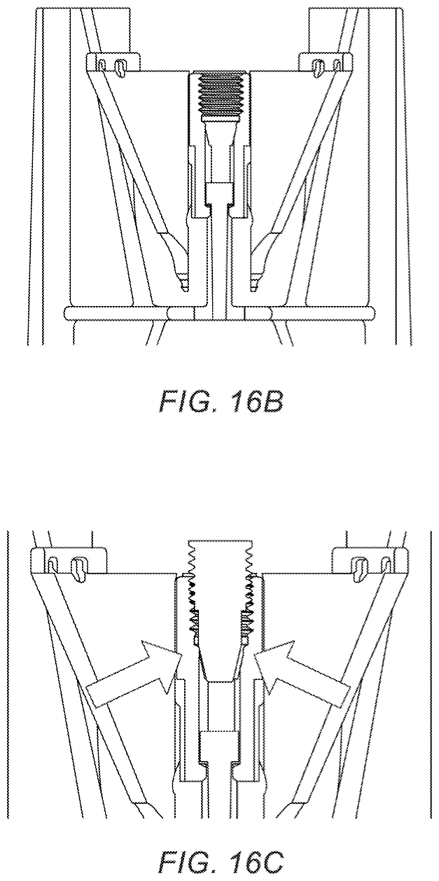

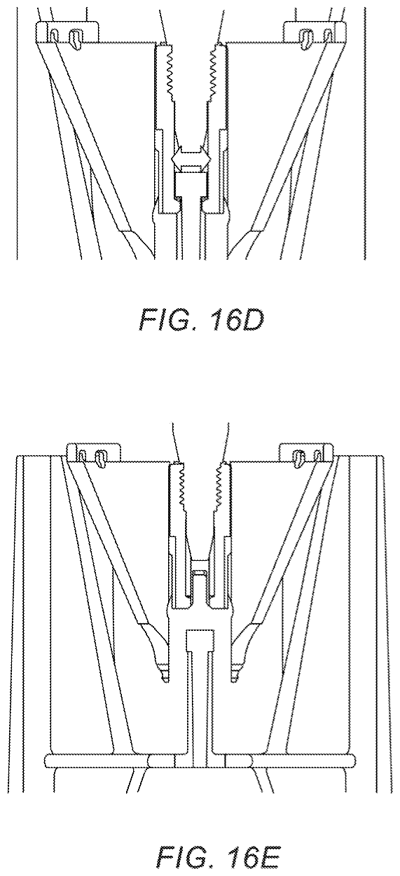

In some embodiments of the present disclosure, top section 212 of heart valve holder 200 may comprise a platform 220 extending from at least one wall of the hollow heart valve holder 200 into the hollow heart valve holder 200. In some embodiments, platform 220 may originate from the anterior side 204 of heart valve holder 200. In other embodiments, platform 220 may originate from the posterior side 208 of heart valve holder 200. In other embodiments, platform 220 may extend from the anterior side 204 to posterior side 208 of heart valve holder. Alternatively, platform 220 may be positioned laterally on heart valve holder 200. Preferably, the platform 220 is level with the top section 212 of heart valve holder 200 and does not protrude above top section 212 of heart valve holder 200, although platform 220 may also protrude above top section 212 of heart valve holder 200 in some embodiments. In some embodiments, platform 220 may comprise a first connector element 224 to connect to a second connector element 728 on handle 700 (see FIGS. 7 to 10). In some embodiments, the first connector element 224 may be a thread hole 224 and the second connector element 728 may be a threaded portion 728, the thread hole 224 adapted to receive threaded portion 728 of handle 700 (see FIGS. 7 to 10). Thread hole 224 may have female threads adapted to accept male threads on threaded portion 728 and may be shaped such that handle 700 is connected to and fastened to heart valve holder 200 by rotating handle 700 clockwise. In some embodiments, platform 220 may have a length of between 12 and 23 mm, and preferably 16.5 mm. In some embodiments, platform 220 may have a thickness of between 5 and 10 mm, and preferably 6.5 mm. In some embodiments, platform 220 may have a width of between 5 and 15 mm, and preferably 8 mm. In some embodiments, thread hole 224 may extend through the thickness of platform 220, although thread hole 224 may terminate within platform 220 in other embodiments (see FIG. 5). In some embodiments, thread hole 224 may have a diameter of between 3 and 8 mm, and preferably 4 mm. In some embodiments, platform 220 may have a protuberance 232 extending below platform 220, protuberance 232 comprising a third connector element 236 to connect to a fourth connector element 1108 on a support structure 1100 (see FIGS. 11 and 12). In some embodiments, the third connector element 236 may be aligned with first connector element 224. In some embodiments, third connector element 236 may be a screw hole 236 and fourth connector element 1108 may be a threaded top 1108 on a pillar 1104, screw hole 236 adapted to receive threaded top 1108 of pillar 1104 of support structure 1100 (see FIGS. 11 and 12). Preferably, screw hole 236 has female threads adapted to accept male threads on threaded top 1108. In some embodiments, screw hole 236 may have a diameter corresponding to that of thread hole 224. Alternatively, screw hole 236 may have a diameter that differs from the diameter of thread hole 224. Screw hole 236 may have a diameter that is larger or smaller than the diameter of thread hole 224. In some embodiments, thread hole 224 may have a diameter of 4.7 mm, and screw hole 236 may have a diameter of 4.7 mm. In some embodiments, the third connector element may comprise extended arms that can be deflected out when fastening the connection between the first connector element and the second connector element (see FIGS. 16B-16E). In such an embodiment, the arms of the third connector element will disengage the third connector element from the fourth connector element allowing the valve holder or valve holder and prosthetic valve to be lifted-off from the support structure. In such an embodiment, it is to be understood that the third connector element may be made of a flexible material that allows the deflection as described above or may be shaped to allow the deflection as described above. In some embodiments, support structure as defined herein may also be termed as a “carrier”.

In some embodiments of the present disclosure, top section 212 of heart valve holder 200 may further comprise at least one anchoring element 240 formed at an upper surface 238 at the top section 212. Anchoring element 240 may be shaped as a block, or any other suitable shape. In some embodiments, there may be three anchoring elements 240a, 240b, and 240c. In some embodiments, there may be a fourth anchoring element 240d. In some embodiments, anchoring elements 240 may extend between 1 and 3 mm, and preferably 2 mm, above upper surface 238 of heart valve holder 200. In other embodiments, anchoring elements 240 may be embedded within top section 212 or the main body of heart valve holder 200. Anchoring elements 240 may each comprise at least one anchoring hole 244. In some embodiments, anchoring element 240a and anchoring element 240b may have two anchoring holes 244 each, and anchoring element 240c may have four anchoring holes 244. In other embodiments, anchoring element 240a, anchoring element 240b, and anchoring element 240c may each have four anchoring holes 244. Preferably, where there are more than one anchoring hole 244 in an anchoring element 240, anchoring holes 244 of the anchoring element 240 are arranged parallel to top section 212 of heart valve holder 200. In some embodiments, there may be a first anchoring element 240a at a 10 o'clock position on heart valve holder 200, a second anchoring element 240b at a 2 o'clock position on heart valve holder 200, and a third anchoring element 240c at a 6 o'clock position on heart valve holder 200. In some embodiments, first anchoring element 240a may be at a position corresponding to the anterolateral commissure of a native mitral valve, second anchoring element 240b at a position corresponding to the posteromedial commissure of a native mitral valve, and third anchoring element 240c may be at a position corresponding to a midpoint of the posterior leaflet of a native mitral valve. In other embodiments, there may be a fourth anchoring element 240d at a position corresponding to a midpoint of the anterior leaflet of a native mitral valve. Preferably, anchoring holes 244 of anchoring elements 240 are adapted to receive a safety suture 320 used to mount the annulus 308 of the prosthetic heart valve 304 to heart valve holder 200 so that the prosthetic heart valve 304 will be fixed securely to heart valve holder 200 (see FIGS. 3A and 3B).

In some embodiments of the present disclosure, bottom section 216 of heart valve holder 200 may comprise at least one leg 248. The at least one leg 248 may have a length of between 5 and 10 mm, and preferably 8 mm. The at least one leg 248 may comprise holes to advantageously reduce the overall weight of heart valve holder 200. Preferably, where there are two legs 248, legs 248 are located on either side of the width of the bottom section 216 of heart valve holder 200 such that apexes 252 of legs 248 align with the location of papillary muscles of the heart. In some embodiments, there may be a distance of between 12 and 24 mm, and preferably 20 mm, between legs 248. In some embodiments, each of legs 248 may align with the location of a papillary muscle in the left ventricle of the heart. In some embodiments, a first leg 248a may align with a posterior papillary muscle of the left ventricle and a second leg 248b may align with an anterior papillary muscle of the left ventricle. In some embodiments, each of the at least one leg 248 may comprise two anchoring holes 244, anchoring holes 244 of the at least one leg 248 adapted to receive safety suture 320 used to mount cords 316 of a prosthetic heart valve 304 to legs 248, therefore allowing cords 316 of prosthetic heart valve 304 to be fixed securely to heart valve holder 200 (see FIGS. 3A and 3B).

FIG. 3A is a schematic illustration of a top view of a prosthetic heart valve 304 mounted on heart valve holder 200, and FIG. 3B is a schematic illustration of a front perspective view of a prosthetic heart valve 304 mounted on heart valve holder 200, in accordance with some embodiments of the present disclosure. A prosthetic heart valve 304 may comprise an annulus 308, leaflets 312 and cords 316. Prosthetic heart valve 304 may be attached to heart valve holder 200 through a safety suture 320 running through prosthetic heart valve 304 and various anchoring holes 244 on heart valve holder 200. Safety suture 320 may be a loop attaching the prosthetic heart valve 304 to heart valve holder 200. In some embodiments, safety suture 320 may run from first anchoring element 240a, through first leg 248a, third anchoring element 240c, second leg 248b, second anchoring element 240b, third anchoring element 240c, and looped back to first anchoring element 240a. In some embodiments, safety suture 320 may first enter annulus 308 of prosthetic heart valve 304 through anchoring hole 244a on first anchoring element 240a, go inwards towards and out of anchoring hole 244b on first leg 248a to secure cords 316 of prosthetic heart valve 304, enter inwards through anchoring hole 244k on first leg 248a, go outward through anchoring hole 244i on third anchoring element 240c to secure annulus 308 of prosthetic heart valve 304, enter inwards through anchoring hole 244h on third anchoring element 240c, go outward through anchoring hole 244f on second leg 248b to secure cords 316 of prosthetic heart valve 304, enter inwards through anchoring hole 244c on second leg 248b, go outward through anchoring hole 244d on second anchoring element 240b to secure annulus 308 of prosthetic heart valve 304, enter inwards through anchoring hole 244c on second anchoring element 240b, go outward through anchoring hole 244g on third anchoring element 240c to secure annulus 308 of prosthetic heart valve 304, enter inwards through anchoring hole 244j on third anchoring element 240c and go outwards through anchoring hole 2441 on first anchoring element 240a to connect with the start of safety suture 320 at anchoring hole 244a at first anchoring element 240a. Preferably, safety suture 320 should be straightened and taut to secure prosthetic heart valve 304 to heart valve holder 200 and prevent movement. Once the prosthetic heart valve 304 has been transplanted into a heart of a patient, the surgeon may retract the entire apparatus 100, along with the safety suture 320 by cutting the safety suture 320.

FIG. 4 is a schematic illustration of a tapering of heart valve holder 200, in accordance with some embodiments of the present disclosure. Heart valve holder 200 may taper at a first gradient X between top section 212 and a midpoint 404 of heart valve holder 200 where legs 248 connect to heart valve holder 200. Heart valve holder 200 may further taper at a second gradient Y between midpoint 404 and bottom section 216 of heart valve holder where the apex 252 of the at least one leg 248 is located. In some embodiments, first gradient X may differ between anterior side 204 and posterior side 208 of heart valve holder 200. In some embodiments, first gradient X may be between about 5° and about 15°, and preferably 10.47°, at anterior side 204 of heart valve holder 200, while first gradient X may be between about 5° and about 15°, and preferably 10.78°, at posterior side 208 of heart valve holder 200. In some embodiments, second gradient Y may differ between anterior side 204 and posterior side 208 of heart valve holder 200. In some embodiments, second gradient Y may be between about 5° and about 25°, and preferably 21.52°, at anterior side 204 of heart valve holder 200, while second gradient Y may be between about 10° and about 30°, and preferably 26.99°, at posterior side 208 of heart valve holder 200. In other embodiments, first gradient X may be the same at anterior side 204 and posterior side 208 of heart valve holder 200. In yet another embodiment, second gradient Y may be the same at anterior side 204 and posterior side 208 of heart valve holder 200.

FIG. 5 is a schematic illustration of a vertical cross-section of a first alternative heart valve holder 200a, in accordance with some embodiments of the present disclosure. Such embodiment is similar to the embodiments of FIGS. 2A and 2B except first alternative heart valve holder 200a comprises a thread hole 224a that terminates within platform 220a and does not extend fully through the thickness of platform 220a, and first alternative heart valve holder 200a does not comprise a protuberance or screw hole. In some embodiments, platform 220a may be 7 mm thick and 7 mm wide, while thread hole 224a may be 5 mm deep, with a “M3×0.5” type configuration.

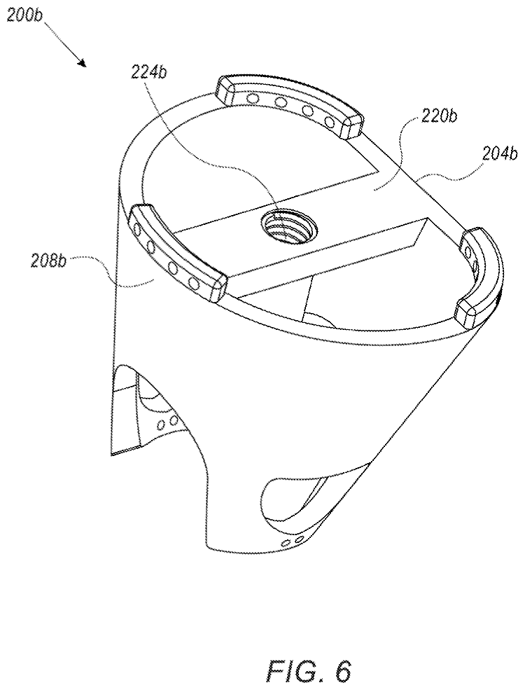

FIG. 6 is a schematic illustration of a top perspective view of a second alternative heart valve holder 200b, in accordance with some embodiments of the present disclosure. Such embodiment is similar to the embodiments of FIGS. 2A and 2B except second alternative heart valve holder 200b comprises a platform 220b that extends from anterior side 204b to posterior side 208b of heart valve holder 200b, and second alternative heart valve holder 200b does not comprise a protuberance or screw hole. In some embodiments, platform 220b may be 5 mm thick and 12 mm wide.

FIG. 7A is a schematic illustration of a front perspective view of a third alternative heart valve holder 200c, in accordance with some embodiments of the present disclosure. FIG. 7B shows a schematic illustration of a top view of the third alternative valve holder 200c. Such embodiment is similar to the embodiments of FIGS. 2A and 2B except platform 220c of the third alternative heart valve holder 200c may have a protuberance 232c extending below platform 220c (see FIG. 7C). As can be seen from FIG. 7C, protuberance 232c comprising a third connector element 236c to connect to a fourth connector element 1108b on a support structure 1100b (see FIGS. 16A-16B). As can be seen from FIG. 7B, the third connector element 236c may be provided in the form of extended arms. Such arms may engage fourth connector element 1108b via a snap fit mechanism since protuberance 232c and extending arms 236c may comprise flexible materials. Before engaging fourth connector element 1108b, the extended arms may bend or deflect (see FIG. 16D) to widen the distance between the tip of the two arms so as to receive fourth connector element 1108b. In some embodiments, other suitable fastening mechanism such as a threaded top for attachment to the third connector element may also be used. In some embodiments, third connector element 236c may be aligned with first connector element 224c. In some embodiments, platform 220c may comprise an opening. The valve holder 200c, in accordance with some embodiments of the present disclosure, may be designed to mimic the outline of the valve prosthesis for holding the prosthesis in place. With this unique and inventive design, the valve holder 200c may advantageously minimize or avoid the risk of slippage of the valve prosthesis from the D-shaped cross-section of the valve holder during implantation of the valve.

FIG. 8 is a schematic illustration of a handle 700, in accordance with some embodiments of the present disclosure. Handle 700 may comprise a solid circular rod with a main body 704, a proximal end 708 adapted to be held by a surgeon and a distal end 712 comprising a second connector element 728 adapted to connect to heart valve holder 200 through first connector element 224 (see FIG. 2A). In some embodiments, handle 700 may comprise a hollow circular rod with a main body 704, a proximal end 708 adapted to be held by a surgeon and a distal end 712 comprising a second connector element 728 adapted to connect to heart valve holder 200 through first connector element 224 (see FIG. 2A). Handle 700 may be made of stainless steel, polypropylene, polyetherketoneketone, acrylonitrile butadiene styrene, Delrin (or polyoxymethylene), or any other similar or suitable material. Handle 700 may have a diameter of between about 5 and about 15 mm, and preferably 8 mm. Handle 700 may have a length of between about 5 and about 20 cm, and preferably 15 cm. In some embodiments of the present disclosure, second connector element 728 may be a threaded portion 728 at distal end 712, threaded portion 728 being complementary to first connector element or thread hole 224 of heart valve holder 200 and adapted to be inserted into thread hole 224 of heart valve holder 200. Threaded portion 728 may have any dimensions depending on the thickness and width of the platform 220.

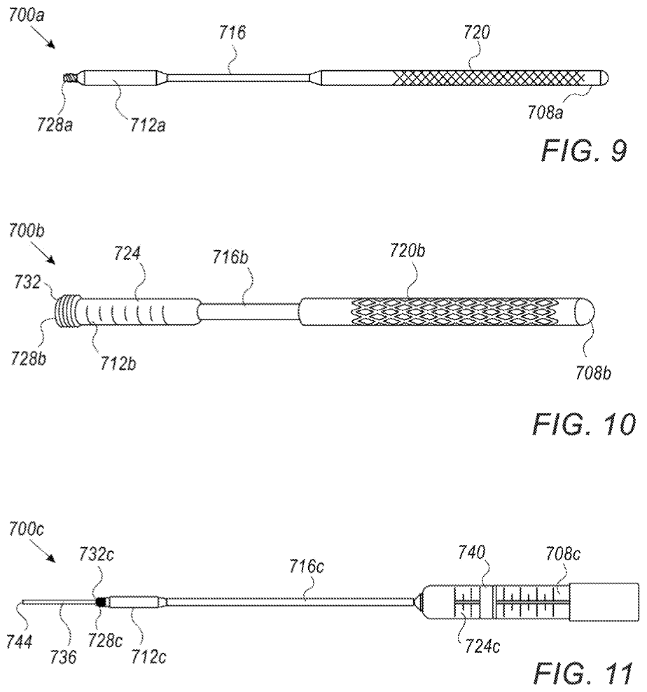

FIG. 9 is a schematic illustration of a first alternative handle 700a, in accordance with some embodiments of the present disclosure. Such embodiment is similar to the embodiment of FIG. 8 except first alternative handle 700a further comprises a flexible portion 716 and a rough portion 720. Flexible portion 716 may be a portion along first alternative handle 700a proximate to distal end 712a of first alternative handle 700a with a narrowed diameter as compared to distal end 712a and proximal end 708a of first alternative handle 700a. In some embodiments, distal end 712a and proximal end 708a of first alternative handle 700a may have a diameter of about 6.2 mm, while flexible portion 716 may have a diameter of about 3.1 mm. In some embodiments, handle 700 may have a length of about 240 mm from proximal end 708a to distal end 712a, and flexible portion 716a may be about 60 mm long and may be located approximately 30 mm from distal end 712a. Flexible portion 716 advantageously allows first alternative handle 700a to be bent, such that a surgeon may bend first alternative handle 700a to their preference and reach difficult angles during surgical procedures. In some embodiments of the present disclosure, first alternative handle 700a further comprises a second connector element 728a, which may be a threaded portion 728a at distal end 712a, threaded portion 728a being complementary to first connector element or thread hole 224 of heart valve holder 200 and adapted to be inserted into thread hole 224 of heart valve holder 200. Threaded portion 728a may have any dimensions depending on the thickness and width of the platform 220.

In some embodiments of the present disclosure, first alternative handle 700a may further comprise a rough portion 720 proximate to proximal end 708a. Rough portion 720 may comprise knurling or check textures to provide friction and enhance a surgeon's grip on first alternative handle 700a. In some embodiments, rough portion 720 may be located about 6.9 mm from the proximal end 708a of first alternative handle 700a, and may have a length of about 80 mm.

FIG. 10 is a schematic illustration of a second alternative handle 700b, in accordance with some embodiments of the present disclosure. Such embodiment is similar to the embodiment of FIG. 9, except second alternative handle 700b comprises a ruler portion 724, thus advantageously eliminating the need for the surgeon to use another measuring tool (a separate and additional tool) to conduct the measurement. Ruler portion 724 may be used to measure any distances required by the surgeon, including the distance between the annulus and the midline of papillary muscles. Ruler portion 724 may be located on distal end 712b of second alternative handle 700b between flexible portion 716b and distal end 712b. Ruler portion 724 may have a length of about 40 mm beginning from the tip of threaded portion 728b. Ruler portion 724 may comprise markings in the form of lines representing linear graduations of a unit of measure (e.g., centimetre or inch) with the marking associated with the number “0” located proximate to distal end 712b. In some embodiments, the markings may account for a distance of threaded portion 728b and begin at a tip 732 of threaded portion 728b. The markings may account for a distance of between about 2 and about 8 mm, and preferably 4 mm. When in use, a surgeon may advantageously guide tip 732 to make contact with papillary muscles of a patient's heart, which will be the start point of measurement. Once tip 732 is in contact with a papillary muscle of the patient's heart, handle 700b should not be moved. A surgeon may then use the ruler portion 724 of handle 700b to take required measurements before proceeding with implantation.

FIG. 11 is a schematic illustration of a third alternative handle 700c, in accordance with some embodiments of the present disclosure. Such embodiment is similar to the embodiment of FIG. 9, except third alternative handle 700c does not have a rough portion and has a channel (not shown) within third handle 700c to accept an inner rod 736. Third alternative handle 700c may have a length of between about 300 and about 360 mm, and preferably 330 mm. The channel (not shown) may run from proximal end 708c through flexible portion 716c, distal end 712c and threaded portion 728c. Proximal end 708c may be shaped as a flat cuboid shape of a width of between about 10 and about 30 mm, and preferably 20 mm, a length of between about 10 and about 50 mm, and preferably 30 mm, and a thickness of between about 1 and about 5 mm, and preferably 3 mm. Proximal end 708c may comprise a ruler section 724c comprising markings in the form of lines representing linear graduations of a unit of measure (e.g., centimetre or inch), with the smallest marking associated with the number “0” located proximate to proximal end 708c. The markings may account for a distance of between about 2 and about 8 mm, and preferably 6 mm. In some embodiments, inner rod 736 may be connected to a sliding portion 740 surrounding ruler section 724c on proximal end 708c. Sliding portion 740 is advantageously adapted to correspond with the markings on ruler section 724c of proximal end 708c and indicate to the surgeon a distance from an end 744 of inner rod 736 to tip 732c of threaded portion 728c. For example, the markings on ruler section 724c of proximal end 708c may be such that when end 744 of inner rod 736 is aligned with tip 732c of threaded portion 728, sliding portion 740 is aligned with a marking on ruler section 724c associated with the number “0”. When sliding portion 740 is moved towards distal end 712c of third alternative handle 700c, inner rod 736 protrudes out of tip 732c of threaded portion 728. The marking on ruler section 724c of proximal end 708c that corresponds with sliding portion 740 represents the distance that end 744 of inner rod 736 is protruding out of tip 732c of threaded portion 728c. For example, a surgeon may align tip 732c of handle 700c with a mitral valve annulus of a patient. The surgeon may then slide the sliding portion 740 of third alternative handle 700c until end 744 of inner rod 736 aligns with the papillary muscles of the patient. This advantageously allows the surgeon to intuitively measure the distance between the papillary muscles and the annulus of the patient.

In some embodiments, flexible portion 716c of third alternative handle 700c may have a narrowed diameter as compared to distal end 712c of third alternative handle 700c. For example, distal end 712c of third alternative handle 700c may have a diameter of about 4 mm, while flexible portion 716c may have a diameter of about 2 mm. Flexible portion 716c may have a length of between about 20 and about 50 mm, and preferably 30 mm and may be located between about 10 and about 20 mm, and preferably 15 mm from distal end 712c.

FIG. 12 is a schematic illustration of a support structure 1100 to support heart valve holder 200, in accordance with some embodiments of the present disclosure. In some embodiments, support structure 1100 may be made partially or entirely of the same material as the valve holder 200 described in the present disclosure. In some embodiments, support structure 1100 may be made of polypropylene, polyetherketoneketone, ABS and Delrin (polyoxymethylene). In some embodiments, said polypropylene, polyetherketoneketone, ABS and Delrin (polyoxymethylene) may be of a medical grade. In some embodiments, said polypropylene, polyetherketoneketone, ABS and Delrin (polyoxymethylene) may be biocompatible polypropylene, polyetherketoneketone, ABS and Delrin (polyoxymethylene). In some embodiments, said biocompatible materials may comply with the US Pharmacopeial Convention (USP) Class VI or ISO 10993-1. FIG. 13 is a schematic illustration of support structure 1100 supporting a heart valve holder 200, in accordance with some embodiments of the present disclosure. FIG. 14 is a schematic illustration of support structure 1100 and heart valve holder 200 inserted into a container 1304 for transport, in accordance with some embodiments of the present disclosure. Support structure 1100 supports heart valve holder 200 within container 1304 to prevent any damage during transportation and accidental drops. Support structure 1100 may be shaped as a hollow cylinder with an inner diameter of between about 40 and about 70 mm, and preferably 50 mm, a wall thickness of between about 1 and about 4 mm, and preferably 1 mm, and a height of between about 40 and about 70 mm and preferably 59 mm. Alternatively, support structure 1100 may be a structure of any shape provided it adequately supports the valve holder 200 within container 1304 (see for example FIG. 16A). In some embodiments, support structure 1100 may be a hollow structure of any shape. Preferably, support structure 1100 is sufficiently wide such that its walls do not contact heart valve holder 200. In some embodiments, support structure 1100 may have at least one opening 1112 along its walls to reduce the weight of support structure 1100.

In some embodiments of the present disclosure, support structure 1100 may comprise a base structure 1116 at a bottom of support structure 1100, base structure 1116 having a length corresponding to a diameter of support structure 1100. In some embodiments, base structure 1116 may have a width of between 5 and 20 mm, and preferably 8 mm, and a thickness of between 2 and 5 mm, and preferably 3 mm. The base structure 1116 may further comprise a fourth connector element 1108. Fourth connector element 1108 may be a threaded top 1108 of vertical pillar 1104 extending from a centre of support structure 1100, threaded top 1108 shaped complementary to screw hole 236 and/or thread hole 224 on heart valve holder 200. Vertical pillar 1104 thus receives heart valve holder 200 and supports heart valve holder 200 (see FIG. 13). Preferably, threaded top 1108 comprises male threads that correspond to female threads of screw hole 236 and/or thread hole 224 and shaped such that heart valve holder 200 is connected to and fastened to support structure 1100 by rotating heart valve holder 200 anti-clockwise.

In some embodiments of the present disclosure, support structure 1100 with heart valve holder 200 may be held in a container 1304 comprising a receptacle 1308 and a lid 1312 (see FIG. 14). Receptacle 1308 and lid 1312 may provide surfaces for labelling. Such labels may include the name of the product, copyrights, and the design. Such labels may further include a quick response code (QR code) that will contain patient information such as patient name and identification number, product details such as size and manufacture date, the QR code assisting with easy tracking of the product. Other labels that may be included are product specifications and manufacturer information. To assemble support structure 1100 and heart valve holder 200 within container 1304, a user may remove lid 1312 of container 1304. The user may then secure handle 700 to heart valve holder 200 by inserting threaded portion 728 into thread hole 224 and rotating handle 700 clockwise. The user may then secure heart valve holder 200 to support structure 1100 by inserting screw hole 236 and/or thread hole 224 into threaded top 1108 and rotating heart valve holder 200 anti-clockwise while holding onto handle 700. The user may remove handle 700 from heart valve holder 200 by rotating handle 700 anti-clockwise. The user may then close container 1304 by securing lid 1312 over receptacle 1308.

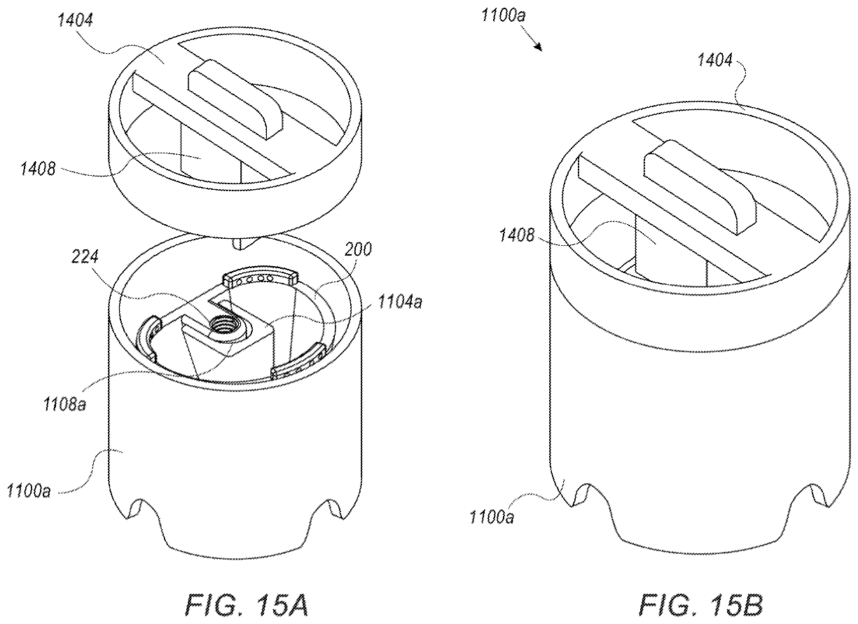

FIG. 15A is a schematic illustration of a first alternative support structure 1100a supporting a heart valve holder 200, and FIG. 15B is a schematic illustration of first alternative support structure 1100a with a cap 1404 closing the support structure 1100a for supporting valve holder 200, in accordance with some embodiments of the present disclosure. Such embodiment is similar to the embodiment of FIG. 12 except fourth connector element 1108a is an indent 1108a on pillar 1104a adapted to receive platform 220 of heart valve holder 200. First alternative support structure 1100a may further comprise a cap 1404. Cap 1404 may be fit onto support structure 1100a such that a bottom edge of cap 1404 and top edge of support structure 1100a are flush against each other. Cap 1404 may comprise an extension 1408, extension 1408 comprising a pin (not shown) that fits into thread hole 224 of heart valve holder 200.

FIG. 16A is a schematic illustration of a second alternative support structure 1100b supporting a heart valve holder 200. As can be seen from FIG. 16A, second alternative support structure 1100b comprises a plurality of separate walls defining an opening for receiving heart valve holder 200. Second alternative support structure 1100b may comprise a fourth connector element to be connected to the third connector element of valve holder 200. In some embodiments, the fourth connector element is a pillar 1104b mounted on a second platform 1116b connecting the plurality of the walls such that fourth connector element 1108b is positioned at the centre of the opening. Each of the plurality of walls that are separated to one another may be provided with a support to stabilize support structure 1100b. As one may appreciate, second alternative support structure 1100b comprising the plurality of separate walls connected by the second platform may form an integrated structure. In some embodiments, the pillar may include a top section having a shape that is complementary to extended arms of third connector element 236c. FIG. 16B illustrates heart valve holder 200 being supported by second alternative support structure 1100b with pillar 1104b. To secure valve holder 200 to second alternative support structure 1100b, a user may align third connector element 236c and fourth connector element 1108b. The user may subsequently rotate the assembly consisting of the handle and the valve holder holding the prosthetic valve clockwise until arms 236c of the third connector element deflect out and consequently receive fourth connector element 1108b to secure valve holder 200 on second alternative support structure 1100b. The user may then rotate handle 700 anti-clockwise to release handle 700 from valve holder 200. To retrieve the prosthetic valve being held by valve holder 200, a user may use handle 700 and connect handle 700 to valve holder 200 by turning the rod clockwise to a fully threaded position (see FIG. 16C). Further turns of handle 700 to the same direction will cause extended arms 236c of the third connector element deflected out (distance between the tip of the extended arms is widened) causing valve holder 200 disengaged from support structure 1100b. The user may then lift valve holder 200 off from support structure 1100b (see FIGS. 16D-16E). To facilitate the bending or deflection of extended arm 236c, such arm may have tapered internal surface. That is the internal surface that may be in contact with second connector element. As mentioned above, the shape of support structure supporting a heart valve holder is not limited to FIG. 16A, support structure may also be provided in other suitable shapes.

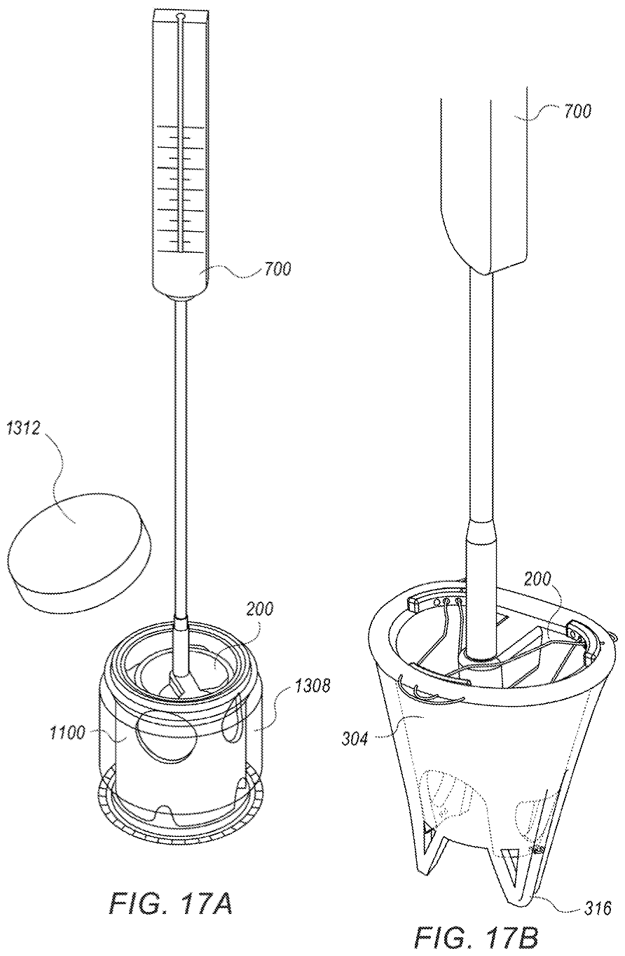

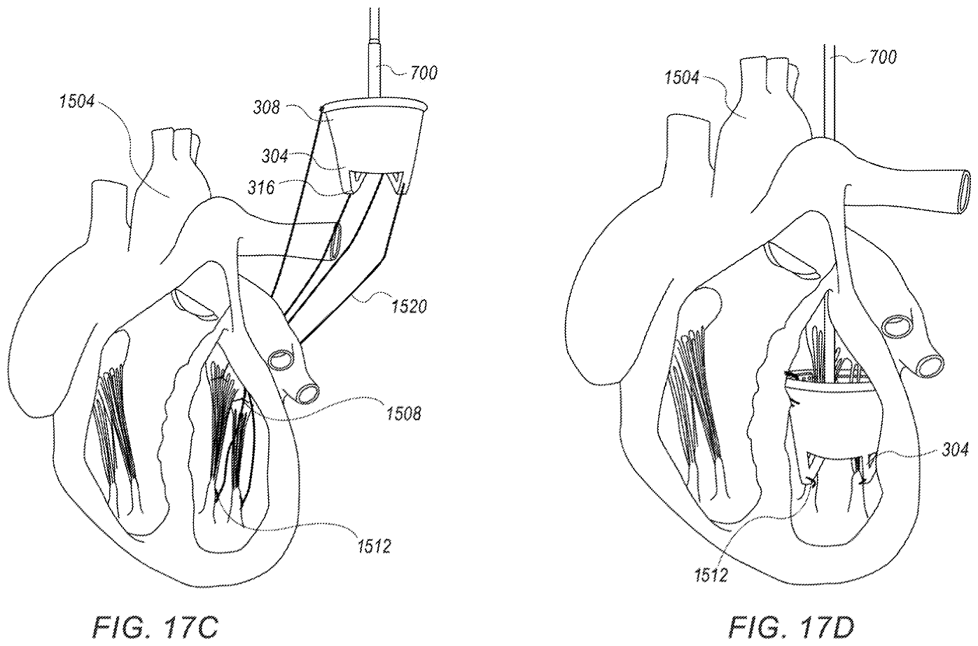

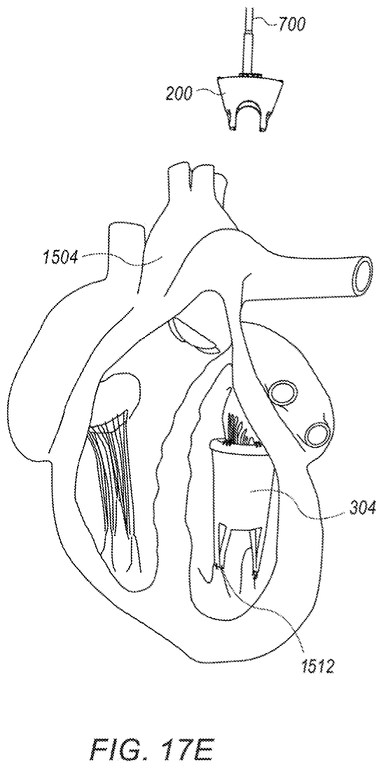

FIGS. 17A, 17B, 17C, 17D and 17E are schematic illustrations of a process of implanting prosthetic heart valve 304 in a heart 1504 of a patient using a heart valve holder 200, in accordance with some embodiments of the present disclosure. Prior to implantation, a practitioner may use embodiments of handle 700b or 700c that comprise ruler portion 724 to measure dimensions of a heart valve and native valve annulus 1508 of a patient to select an appropriate valve for implantation. The practitioner may also use ruler portion 724 to measure an annulus height to adjust the length of cords 316 on prosthetic heart valve 304. Prior to implanting the selected prosthetic heart valve, the practitioner may select a heart valve holder 200 by determining an annular size of a heart valve of a patient receiving the prosthetic heart valve. The practitioner may also use prolene sutures to mark specific landmarks on the native valve annulus 1508 of the patient to assist in the orientation of and subsequent attachment of annulus 308 of prosthetic heart valve 304 to the native valve annulus 1508 of the patient. In some embodiments, the landmarks for a mitral valve annulus may be the right and left commissures and the mid-point of the posterior annulus, i.e., a 2 o'clock position on a mitral valve annulus corresponding to the right fibrous trigone, a 10 o'clock position on a mitral valve annulus corresponding to the left fibrous trigone, and a 6 o'clock position on a mitral valve annulus corresponding to a midpoint of the posterior annulus. In some embodiments, the landmarks for a mitral valve annulus may be a 2 o'clock position on a mitral valve annulus corresponding to the right fibrous trigone, a 4 o'clock position on the a mitral valve annulus corresponding to a point between the middle and the posterior scallops of the posterior leaflet, an 8 o'clock position on a mitral valve annulus corresponding to a point between the anterior and the middle scallops of the posterior leaflet of a mitral valve, and a 10 o'clock position on a mitral valve annulus corresponding to the left fibrous trigone.

As shown in FIG. 17A, lid 1312 is removed from receptacle 1308. The practitioner may connect handle 700 to heart valve holder 200 by inserting threaded portion 728 of handle 700 into thread hole 224 and rotating handle 700 clockwise. The practitioner may continue rotating handle 700 clockwise to rotate heart valve holder 200 to remove heart valve holder 200 from support structure 1100.

As shown in FIG. 17B, the practitioner may remove apparatus 100 comprising heart valve holder 200 and handle 700 from receptacle 1308 and attach, mount, or secure a prosthetic heart valve 304 to heart valve holder 200. Prosthetic heart valve 304 may be attached, mounted or secured onto heart valve holder 200 with a safety suture 320 as described above.

As shown in FIG. 17C, the practitioner may use pledgeted gore-tex suture with a double arm needle to place a horizontal mattress stitch from a tip of a corresponding papillary muscle 1512 within approximately 5 mm of the tip to a corresponding cord 316 of the prosthetic heart valve 304. The practitioner may further place a horizontal mattress stitch from a native valve annulus 1508 of the patient to a corresponding position on annulus 308 of the prosthetic heart valve 304. The practitioner may place at least one suture wires 1520 to connect heart 1504 of the patient to the prosthetic heart valve 304 through a designated surgical opening (not shown) on heart 1504 of the patient. In some embodiments, the practitioner may place four suture wires 1520: two suture wires 1520 connecting papillary muscles 1512 to their corresponding cords 316 on prosthetic heart valve 304, and two suture wires 1520 connecting the native valve annulus 1508 to annulus 308 of prosthetic heart valve 304. Preferably, the two suture wires 1520 connecting the native valve annulus 1508 to annulus 308 of prosthetic heart valve 304 are connected to the two commissural points on the native valve annulus 1508.

As shown in FIG. 17D, the practitioner may use a gentle gliding manoeuvre to slide prosthetic heart valve 304 along the at least one suture wire 1520 and insert apparatus 100 and prosthetic heart valve 304 into heart 1504 through the designated surgical opening (not shown). The practitioner may tighten the at least one suture wire 1520 connected to the cords and papillary muscles, tie the at least one suture wire 1520 with dead knots to secure prosthetic heart valve 304 in place, and cut off any excess suture wire 1520. Optionally, the practitioner may secure cords 316 to papillary muscles 1512 using hand-ties. The practitioner may tighten the at least one suture wire 1520 connecting the annulus 308 of prosthetic heart valve 304 to the native valve annulus 1508 of the patient together to securely attach the annulus 308 of prosthetic heart valve 304 in place. Optionally, the practitioner may use prolene sutures making specific landmarks on the native valve annulus 1508 of the patient to orientate annulus 308 of prosthetic heart valve and secure the prosthetic heart valve 304 at the native valve annulus 1508.

As shown in FIG. 17E, after the prosthetic heart valve 304 has been implanted in heart 1504 of the patient, the practitioner may cut safety suture 320 at any visible location to remove safety suture 320. The practitioner may then withdraw apparatus 100 from the heart 1504 of the patient through a designated surgical opening (not shown). The practitioner may further tie knots onto tips of papillary muscles 1512 so the cords 316 are now affixed to the papillary muscles. The practitioner may then pass the commissural stitches through the entire circumference of the native valve annulus 1508 and annulus 308 of the prosthetic heart valve 304 and tied together where they meet. Preferably, the commissural stitches come continuously from each side.

It should be appreciated that the above-described methods and apparatus may be varied in many ways, including omitting or adding steps, changing the order of steps and the type of devices used. It should be appreciated that different features may be combined in different ways. In particular, not all the features shown above in a particular embodiment are necessary in every embodiment of the disclosure. Further combinations of the above features are also considered to be within the scope of some embodiments of the disclosure.

It will be appreciated by persons skilled in the art that the present invention is not limited to what has been particularly shown and described hereinabove. Rather the scope of the present invention is defined only by the claims, which follow.

1. A heart valve holder for holding a prosthetic heart valve, the heart valve holder comprising:
a hollow structure having a cross-section that mimics a shape of an annulus of the prosthetic heart valve, the hollow structure comprising an upper surface;
a platform extending from at least one wall of the hollow structure, the platform comprising a first connector element; and
at least one anchoring element formed at the upper surface of the hollow structure, the at least one anchoring element comprising at least one anchoring hole for suturing the prosthetic heart valve to the heart valve holder.

2. The heart valve holder of claim 1, wherein the cross-section is D-shaped to mimic the shape of the annulus of the prosthetic heart valve, the annulus of the prosthetic heart valve mimicking a shape of a mitral valve annulus.

3. The heart valve holder of claim 1, wherein the heart valve holder is dimensioned to fit within the prosthetic heart valve.

4. The heart valve holder of claim 1, wherein the hollow structure is a tapered tube, the tapered tube tapering from a top section of the hollow structure to a bottom section of the hollow structure.

5. The heart valve holder of claim 1, wherein the at least one anchoring element is positioned corresponding to an anterolateral commissure, a posteromedial commissure, a midpoint of a posterior leaflet, or a midpoint of an anterior leaflet.

6. The heart valve holder of claim 1, further comprising at least one leg at the bottom section of the hollow structure, the at least one leg comprising at least one anchoring hole for suturing of the prosthetic heart valve to the heart valve holder.

7. The heart valve holder of claim 6, wherein the at least one leg is positioned corresponding to a papillary muscle of a left ventricle of a heart.

8. The heart valve holder of claim 1, further comprising a handle, the handle comprising a second connector element to be connected to the first connector element.

9. The heart valve holder of claim 8, wherein the handle comprises a rod.

10. The heart valve holder of claim 8, wherein the handle comprises a flexible section.

11. The heart valve holder of claim 8, wherein the handle comprises a ruler section.

12. The heart valve holder of claim 8, wherein the second connector element comprises a threaded portion for attachment to the first connector element.

13. The heart valve holder of claim 8, wherein the platform further comprises a third connector element.

14. The heart valve holder of claim 13, further comprising a support structure, the support structure comprising a fourth connector element to be connected to the third connector element.

15. The heart valve holder of claim 14, wherein the fourth connector element comprises a threaded top for attachment to the third connector element.

16. The heart valve holder of claim 14, wherein the fourth connector element comprises an indent to receive the third connector element.

17. The heart valve holder of claim 14, wherein the third connector element comprises extended arms for securing the fourth connector element.

18. A method of implanting a prosthetic heart valve, the method comprising:
(i) providing a heart valve holder for holding the prosthetic heart valve, the heart valve holder comprising:
a hollow structure having a cross-section that mimics a shape of an annulus of the prosthetic heart valve, the hollow structure comprising an upper surface;
a platform extending from at least one wall of the hollow structure, the platform comprising a first connector element; and
at least one anchoring element formed at the upper surface of the hollow structure, the at least one anchoring element comprising at least one anchoring hole for suturing the prosthetic heart valve to the heart valve holder:
(ii) providing a handle, the handle comprising a second connector element;
(iii) connecting the handle to the heart valve holder;
(iv) mounting the prosthetic heart valve on the heart valve holder;
(v) attaching at least one suture wire between a heart and the prosthetic heart valve; and
(vi) sliding the prosthetic heart valve along the at least one suture wire into the heart.

19. The method of claim 18, wherein mounting the prosthetic heart valve comprises using a single suture to attach the prosthetic heart valve to the heart valve holder.

20. The method of claim 18, further comprising using a ruler portion on the handle to take measurements of the heart.

21. The method of claim 18, further comprising:
(vii) positioning the prosthetic heart valve in the heart using marked landmarks on a native valve annulus; and
(viii) tying the at least one suture wire to attach the prosthetic heart valve to the heart.
